(19) United States
(12) Patent Application Publication
(10) Pub. No.: US 2012/0121834 A1
(43) Pub. Date: May 17, 2012

(54) SILK PRODUCT AND METHOD OF MAKING THE SAME

(75) Inventors: Chun-Lung Wang, Nantou County (TW); Kao-Ming Chen, Nantou County (TW)

(73) Assignee: National Taiwan Craft Research and Development Institute, Nantou County (TW)

(21) Appl. No.: 12/946,567

(22) Filed: Nov. 15, 2010

(51) Int. Cl. B32B 1/02; B27J 1/02; B05D 3/00; A01K 67/04

(52) U.S. Cl. 428/35.6; 427/325; 144/333; 119/270

(57) ABSTRACT

A silk product includes a hollow bamboo structure, a bamboo covering layer formed on outer surfaces of the hollow bamboo structure and a silk covering layer securely covering outer surfaces of the bamboo covering layer. With such a structure, the silk product is able to withhold load without any deformation.

### BACKGROUND OF THE INVENTION

1. Field of the Invention

The present invention relates generally to a silk product, and more particularly to a silk product composed of a base, multiple bamboo strips one end of which are respectively and securely connected to the base and the other end of which are connected to a connector to form a hollow structure, a bamboo covering layer formed by fine bamboo strips knitted together and a silk layer covering on top of the bamboo covering layer.

2. Description of the Prior Art

Human has been using silk to make things we use almost every day for centuries. As known in record, as well as in practice, silk is a natural substance that contaminates nothing to our environment and has features nothing artificially made can compete. First of all, silk made clothes has excellent feature for keeping warm. Normally when silk made clothes or the like is made, there are multiple layers of silk superposed on one another. Because there are voids existing between layers, heat is perfectly preserved when a user is wearing this silk made clothes or the like. So therefore, the silk made clothes has great heat preservation effect. Still further, due to the existence of the voids between layers of silk, moisture may easily flow through the layers of silk to make the user feel comfortable with regard to humidity. One more feature of the silk made clothes has is also because of the existence of the voids between silk layers. Due to the voids, acoustic sound is absorbed. In another word, the silk made clothes or product has soundproof effect to a certain degree. Lastly but not the least is that the silk is full of protein fiber such that when the silk made product is in contact with human skin, there is a great compatibility. Also because the silk made product has great texture and is delicate, when a user is using the silk product, the user will feel comfortably.

Despite all the advantages the silk product has, the silk product is still very limited to two dimensional product and seldom applied to different fields. Since silk is soft and although its elasticity is excellent, the silk product is still breakable and cannot withstand impact from outer sources.

### SUMMARY OF THE INVENTION

To address these and other objects and in view of its purposes, the present invention provides a silk product composed of multiple bamboo strips end of which are securely connected to a base and the other end of which are connected to a connector to form a hollow structure, a bamboo covering layer formed on outer surfaces of the hollow bamboo structure and by fine bamboo strips knitted together and a silk layer covering top of the bamboo covering layer.

In accordance with another objective of the present invention, a method for fabricating the silk product includes the steps of forming a hollow bamboo structure, forming a bamboo covering layer on outer surfaces of the hollow bamboo structure, and forming a silk covering layer on outer surfaces of the bamboo covering layer.

In accordance with the objective of the present invention, the bamboo strips may undergo a heating process to make the bamboo strips bent.

In accordance with still another objective of the present invention, the bamboo covering layer is made of fine bamboo strips knitted together.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a perspective view of the silk product of the present invention;

FIG. 2 is a cross sectional view of the silk product of the present invention;

FIG. 3 shows a flow process of making the silk product of the present invention;

FIG. 4 is an exploded perspective view of a partial structure of the silk product of the present invention;

FIG. 5 is a perspective view showing that the bamboo covering layer is formed on outer surfaces of the hollow bamboo structure as shown in FIG. 4;

FIG. 6 is a schematic view showing an embodiment of forming the silk covering layer on outer surfaces of the bamboo covering layer; and

FIG. 7 is a schematic view showing a different embodiment of the silk product of the present invention.

It should be noted that all the Figures are diagrammatic. Relative dimensions and proportions of parts of the drawings have been shown exaggerated or reduced in size, for the sake of clarity and convenience in the drawings. The same reference signs are generally used to refer to corresponding or similar features in modified and different embodiments.

### DETAILED DESCRIPTION OF THE PREFERRED EMBODIMENT

In the following description, numerous specific details are given to provide a thorough understanding of the invention. However, it will be apparent to one skilled in the art that the invention may be practiced without these specific details. In order to avoid obscuring the present invention, some well-known system configurations and process steps are not disclosed in detail.

Likewise, the drawings showing embodiments of the apparatus are semi-diagrammatic and not to scale and, particularly, some of the dimensions are for the clarity of presentation and are shown exaggerated in the figures. Also, where multiple embodiments are disclosed and described having some features in common, for clarity and ease of illustration and description thereof, like or similar features one to another will ordinarily be described with like reference numerals.

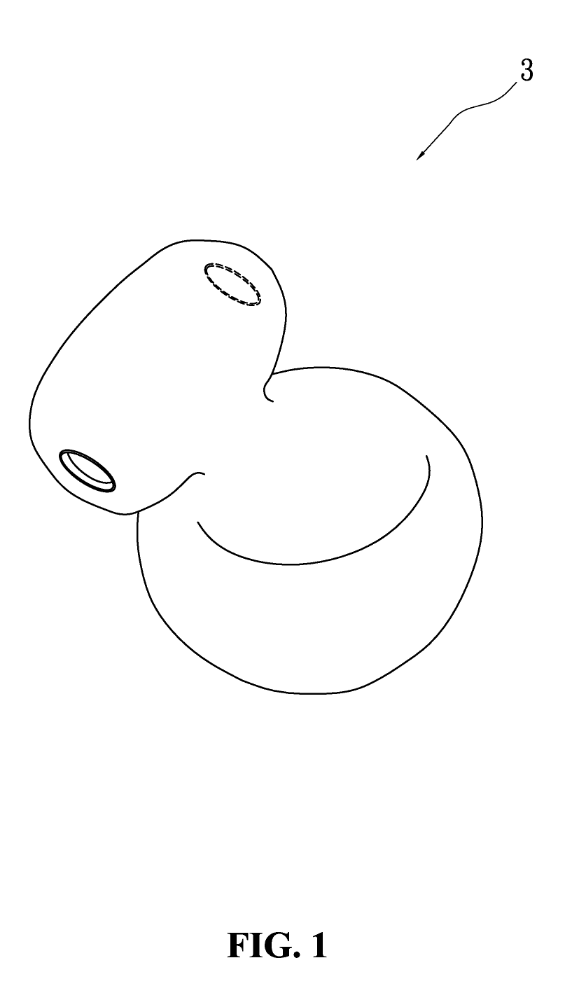

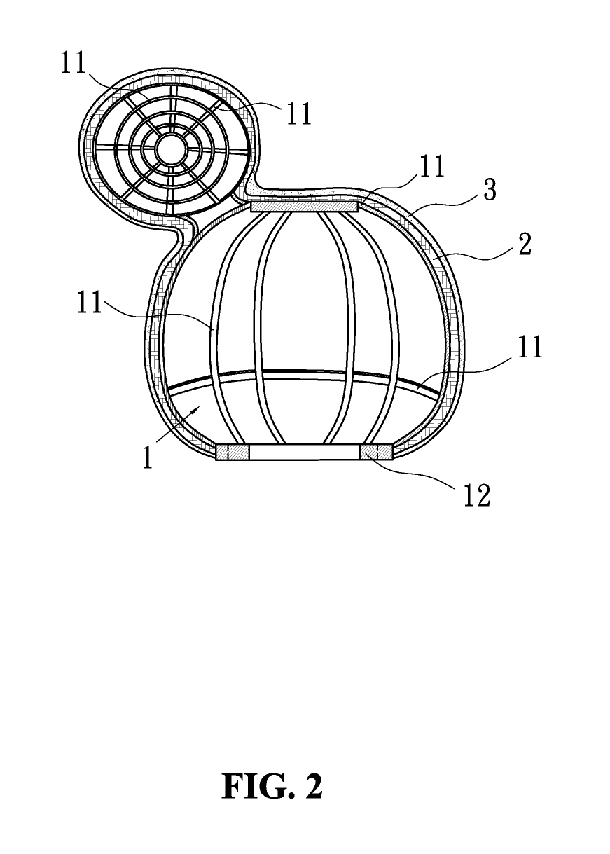

With reference to FIGS. 1 and 2, it is to be noted that the silk product constructed in accordance with one embodiment of the present invention is consisted of multiple bamboo strips 11 one end of which are securely connected to a base 12 and the other end of which are connected to a connector 14 to form a bamboo hollow structure 1. It is noted that each of the bamboo strips 11 is bent to a predetermined curvature so as to fit the designated shape of the bamboo hollow structure 1. A bamboo covering layer 2 made of fine bamboo strips respectively knitted together is formed on outer surfaces of the hollow bamboo structure. After the bamboo covering layer 2 is constructed, glue may be applied to fix relative positions of the respectively knitted bamboo strips 11.

A silk covering layer 3 is securely applied on outer surfaces of the bamboo covering layer 2. The application of the silk covering layer 3 may adopt different suitable methods. For example, the manufacturer may plant live worms on the top surfaces of the bamboo covering layer 2 to allow the worm to produce silk on the surfaces of the bamboo covering layer 2.

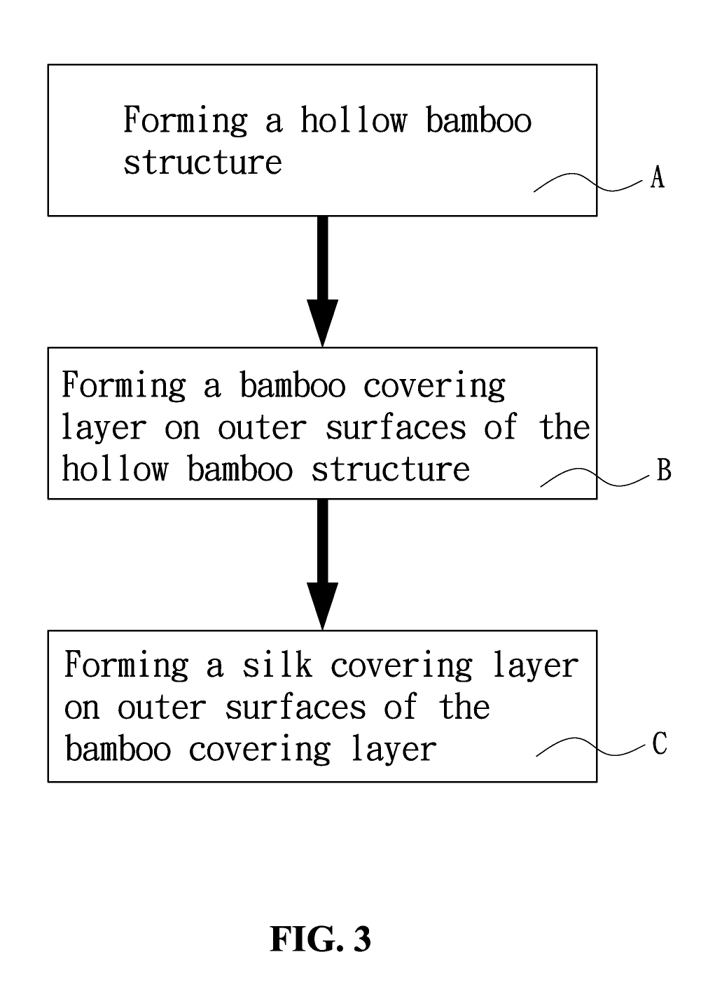

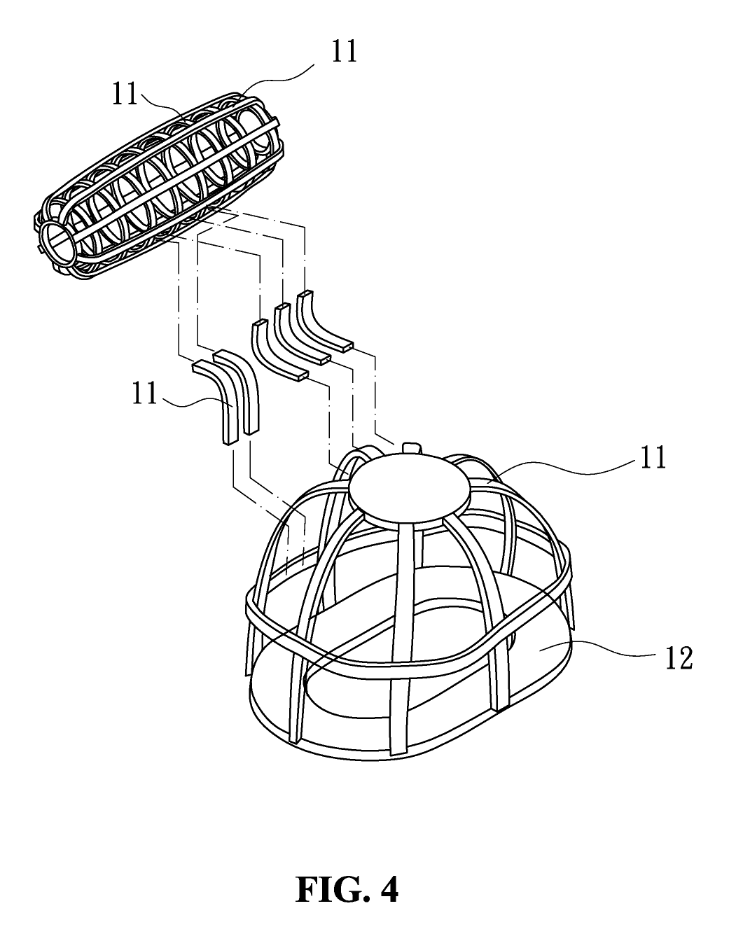

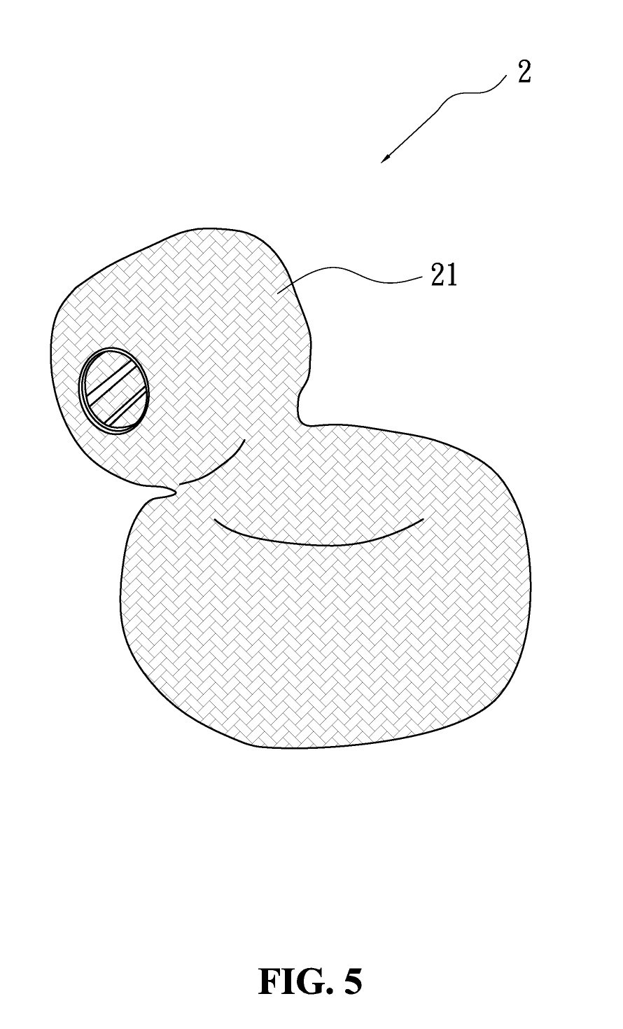

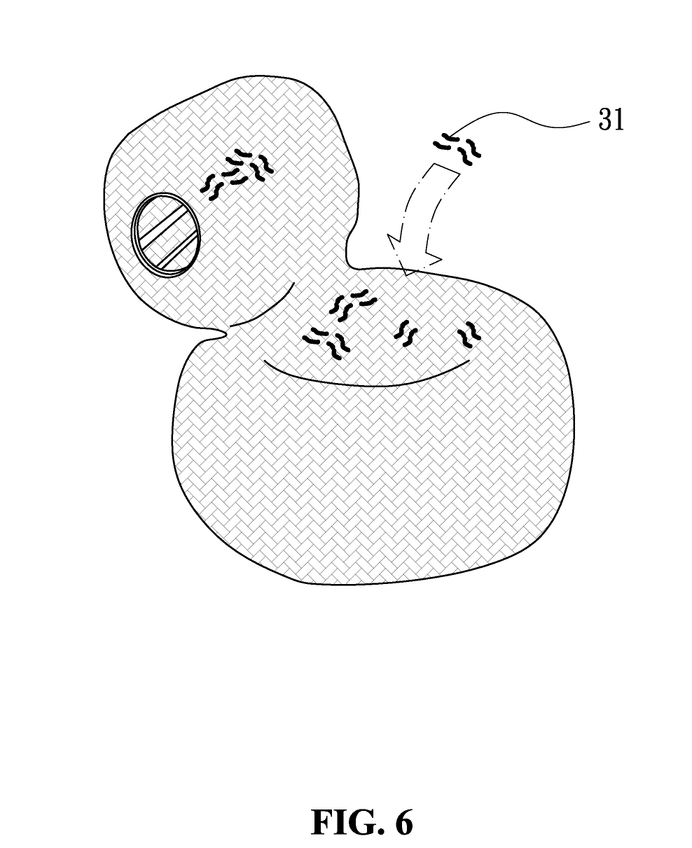

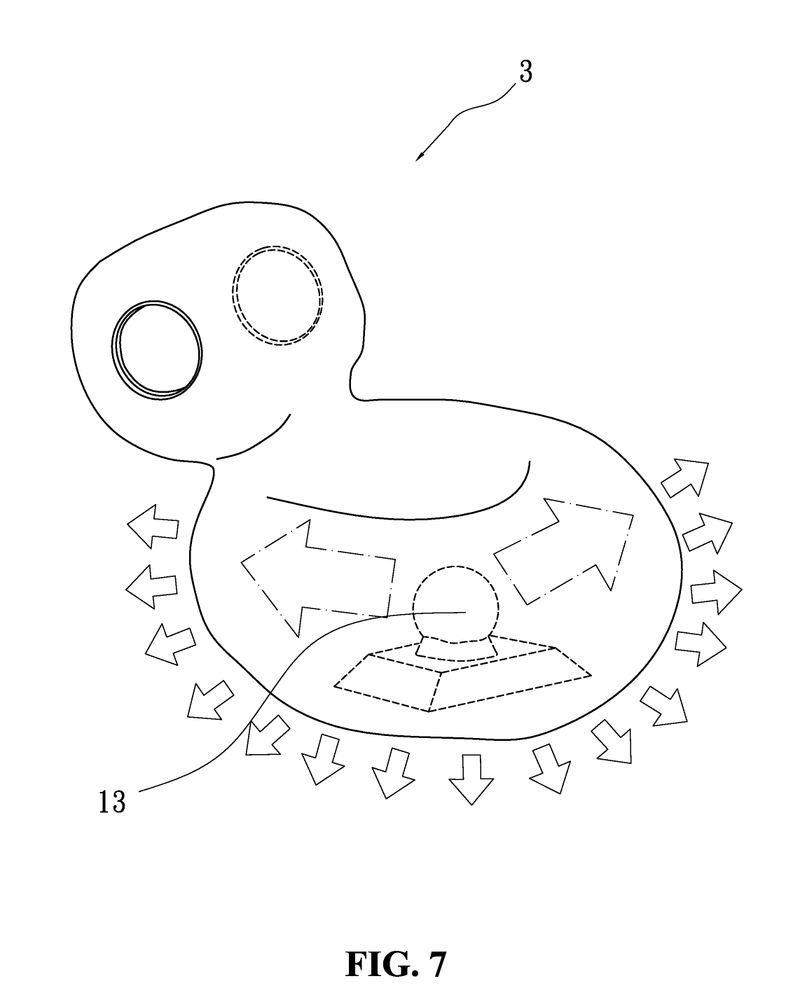

With reference to FIGS. 2-7, the method of one embodiment of the present invention is shown to have steps of forming a hollow bamboo structure, forming a bamboo covering layer on outer surfaces of the hollow bamboo structure, and forming a silk covering layer on outer surfaces of the bamboo covering layer. The following description will aim at each specific step to explain how each step is processed.

In the step of forming a hollow bamboo structure (A), a base 12 is prepared. Then multiple bamboo strips 11 are prepared. Each one end of the bamboo strips 11 is securely connected to the base 12 and the other end of the bamboo strips 11 is then connected to a connector 14. It is to be noted that before the bamboo strips 11 are connected to the connector 14, each of the bamboo strips 11 may go through a heating process to allow the bamboo strips 11 to bend to a predetermined degree so as to fit a specific shape requirement of the silk product. After the bamboo strips 11 are bent and fixed to the connector 13, a hollow bamboo structure is formed.

In the step of forming a bamboo covering layer 2 (B), multiple bamboo strips 21 (different from those used in the hollow bamboo structure forming step) are knitted to form a nicely knitted web-like covering. The web-like covering is then securely placed on outer surfaces of the hollow bamboo structure to cover gaps between two adjacent bamboo strips 11.

In the step of forming a silk covering layer (C), multiple worms (31) are planted on outer surfaces of the bamboo covering layer 2 to allow the worms 31 to produce silk to completely cover the bamboo covering layer 2. The step of silk covering layer formation may be repeated several times till the thickness of the silk covering layer 3 reaches the predetermined level.

The method of the present invention may also include a step of coloring outer surfaces of the outermost silk covering layer. That is, the manufacturer may choose different colors to increase the appealing effect of the final product.

In addition, an illuminating element 13, such as an LED, a light bulb or the like, is placed inside the hollow bamboo structure so that when the final product is use, the illumination of the illuminating element 13 may display the diversity of silk covering layer 3.

Because there is a hollow bamboo structure as a base, the final silk product in accordance with the present invention is able to have better load capability than the conventional structure. As a conclusion, the silk product of the present invention fuses the conventional craft art with modern fashion design, which not only gives a new life to the conventional craft, but also improves the visual effect to the completed silk product.

From the disclosure of the present invention, it is notable that the silk product constructed in accordance with the present invention is a breakthrough of a conventional structure and expand the application possibility of conventional silk structure.

Those skilled in the art will readily observe that numerous modifications and alterations of the device and method may be made while retaining the teachings of the invention.

1. A silk product comprising:
a hollow bamboo structure;
a bamboo covering layer formed on outer surfaces of the hollow bamboo structure; and
a silk covering layer securely covering outer surfaces of the bamboo covering layer.

2. The silk product as claimed in claim 1, wherein the hollow bamboo structure comprises:
a base; and
multiple bamboo strips one end of which are securely connected to the base and the other end of which are connected to a connector.

3. The silk product as claimed in claim 1, wherein the bamboo covering layer is made of multiple fine bamboo strips and the multiple fine bamboo strips for making the bamboo covering layer are knitted together to form a web-like covering.

4. The silk product as claimed in claim 2, wherein the bamboo covering layer is made of multiple bamboo strips different from those for the hollow bamboo structure and the multiple bamboo strips for making the bamboo covering layer are knitted together to form a web-like covering.

5. The silk product as claimed in claim 3, wherein the silk covering layer is colored.

6. The silk product as claimed in claim 4, wherein the silk covering layer is colored.

7. A method for making a silk product comprising the steps of:
forming a hollow bamboo structure;
forming a bamboo covering layer on outer surfaces of the hollow bamboo structure; and
forming a silk covering layer on outer surfaces of the bamboo covering layer.

8. The method as claimed in claim 7 further comprising a heating process to each of the bamboo strips for making the hollow bamboo structure to allow the bamboo strips to bend to a predetermined degree.

9. The method as claimed in claim 7, wherein the bamboo covering layer is made of multiple fine bamboo strips knitted together.

10. The method as claimed in claim 8, wherein the bamboo covering layer is made of multiple fine bamboo strips knitted together.

11. The method as claimed in claim 7 further comprising a color coating process to color the silk covering layer.

12. The method as claimed in claim 8 further comprising a color coating process to color the silk covering layer.

13. The method as claimed in claim 9 further comprising a color coating process to color the silk covering layer.

14. The method as claimed in claim 10 further comprising a color coating process to color the silk covering layer.
